(19) United States
(12) Patent Application Publication
(10) Pub. No.: US 2024/0366906 A1
(43) Pub. Date: Nov. 7, 2024

(54) VAPOR BOTTLE NOZZLE ADAPTER FOR CONNECTING FORCED AIR STYLE VAPORIZERS TO STANDARD BOTTLE OR CONTAINER

(71) Applicant: ROY J. STRAUSS, DEERFIELD, IL (US)

(72) Inventor: ROY J. STRAUSS, DEERFIELD, IL (US)

(21) Appl. No.: 18/654,960

(22) Filed: May 3, 2024

Related U.S. Application Data
(60) Provisional application No. 63/557,837, filed on Feb. 26, 2024.
(60) Provisional application No. 63/463,770, filed on May 3, 2023.

(51) Int. Cl. A61M 16/18

(52) U.S. Cl. CPC A61M 16/18

(57) ABSTRACT

The present disclosure is a vapor bottle nozzle adapter that addresses issues and problems with the PZR-style vaporizers, including unpleasant odors and tastes from the silicone components, and the limitation or need to use receptacles that are proprietary and specially made for each PZR-style vaporizer device, by using, a disk component, which sits on top of the PZR-style cover and a specialized tube device, which fits into the disk component, and from which the vapor is expelled downwardly into the bottle or container. Alternatively, a third component, a modified insert component that fits into the PZR vaporizer well instead of the existing cover is used for the assembly. These two or three components work together to allow the vaporizer device to produce better tasting vapor and distribute the vapor from the vaporizer to a non-proprietary receptacle.

### PRIORITY STATEMENT

The present application claims the benefit of U.S. Provisional Patent Application No. 63/463,770, titled VAPOR BOTTLE NOZZLE ADAPTER APPARATUS, SYSTEM AND METHODS THEREOF, filed May 3, 2023, and U.S. Provisional Patent Application No. 63/557,837, titled VAPOR BOTTLE NOZZLE ADAPTER FOR CONNECTING FORCED AIR STYLE VAPORIZERS TO STANDARD BOTTLE OR CONTAINER, filed Feb. 26, 2024, the foregoing applications incorporated by reference herein.

### TECHNICAL FIELD

The present disclosure relates to vaporizer devices used in the process of producing or manufacturing e-cig vapor, CBD, and cannabis products (flower, oil, concentrates, etc.), ultimately for human inhalation. The present disclosure is especially useful for producing the products without the silicone odors found in current vaporizer devices. The present disclosure also allows for the creation of custom receptacles for the end product from well-known bottles and containers or from unique bottles and containers.

### BACKGROUND

There are many existing vaporizer devices on the market in various forms for inhalation of vapor from various substances, such as e-cigarettes, CBD oil, and cannabis concentrates and plant material. One category of such vaporizer devices is the “desktop” vaporizers, meant to be used on a table or desk. Among these desktop vaporizers is a category called “forced air” vaporizers. These Forced Air Style (FAS) vaporizers push air through the mechanism, expelling vapor automatically, thereby not requiring the user to suck air through the vaporizer mechanism.

Among these FAS-type vaporizers are several models which are very similar. These include the Pulsar “Sipper,” the Zenco “Flow” and “Duo,” and Randy's “Loov” vaporizer models, collectively referred to herein as the Pulsar-Zenco-Randy's or “PZR-style” or “PZR” vaporizer devices. These three types of vaporizer devices are very similar to each other in size, style, and operation. They each consist of a base unit that emits vapor vertically from a base unit, through a silicone nozzle, into a special matching glass receptacle. These three PZR-style vaporizers operate the same as each other, but have different external housing and are packaged with slightly different components, such as glass receptacles, accessories, etc.

These PZR-style vaporizers are used by placing them on a table or desk, where they emit vapor in a vertical direction from either a 510-threaded oil cartridge, or an atomizer bowl which may contain oil, plant matter, or other substances which emit vapor when heated. The vapor then travels through a special silicone nozzle, and into a glass receptacle from which the vapor is inhaled by the user.

These vaporizers consist of three main components, the vaporizer base, the silicone nozzle, and the glass receptacle. There are two additional aspects of these vaporizers. The base unit contains a cover, sometimes magnetic, which covers the atomizer and baseplate. This cover fits into a round opening, or a well, which is approximately 2″ wide. The cover is easily removed to expose the well in order to refill the atomizer or replace the 510 cartridge. The cover has a raised round hole in the center, from which the vapor is emitted.

Additionally, the silicone nozzle is composed of two silicone pieces, a stem, and a bell. The bell sits on top of the stem and directs the vapor into the base of the glass receptacle, so that the vapor is not discharged straight up and out of the receptacle.

The standard PZR-style vaporizer devices use a proprietary connector nozzle made of silicone with a special shape requiring a matching glass receptacle. This design creates unpleasant odors, as the silicone material retains odors, which makes them unpleasant to use with normal usage. Cleaning them is difficult and replacements are sold by the manufacturers, which also increases the time and cost of using the underlying vaporizer device.

The standard PZR-style vaporizer devices use existing silicone pieces that come with the purchase of the vaporizer device or can be purchased separately. The silicone devices are shaped to grasp the bottom of the glass receptacle with a groove which requires the flexibility of silicone to position. As such, the hole in the bottom of the receptacle is round with a narrow, tapered lip to accept the groove in the silicone nozzle. The special interaction of the silicone nozzle and the glass receptacle fit together prevent the use of non-proprietary receptacles. In order to work properly, the vaporizer device must be used with the container provided. The vaporizer manufacturers offer a very limited selection of compatible receptacles. This is a problem for people who would like to make their own bottles/receptacles for use with a PZR vaporizer.

These existing vaporizer devices utilize the silicone nozzle fitting as dictated by the receptacle design. Being made of silicone, they accumulate odors, which are imparted to the vapor making it unpleasant to some users and interfering with the purity and taste of the vapor being consumed. Also, being silicone, they are difficult to clean of the odor, so the user must continually purchase new silicone nozzles.

These existing vaporizer devices also use specific silicone fittings which require a specially shaped bottle design for the bottle base and hole. This reduces the use of other receptacles besides the original ones molded specifically for this purpose.

What is needed is a vaporizer device that, among other issues, addresses issues with the current style of vaporizer devices, namely an unpleasant odor and taste silicone components and the limitation of using proprietary glass receptacles. The present invention addresses these and other issues.

### SUMMARY

Vaporizer devices exist that are used to convert CBD, cannabis, and other products into vapor for human inhalation. The present disclosure, a Vapor Bottle Nozzle Adapter, addresses issues and problems with the PZR-style vaporizers, including unpleasant odors and tastes from the silicone components, and the limitation or need to use receptacles that are proprietary and specially made for each PZR-style vaporizer device, and silicone nozzles used in those vaporizer devices. The present disclosure allows users to use the existing vaporizer devices with non-silicone components and manufacture their own container or receptacle regardless of the components that were sold with the vaporizer device.

This disclosure and the Vapor Bottle Nozzle Adapter invention consists of two or three pieces, depending on the configuration. For the three-piece adapter, the disclosure consists of a modified insert component that fits into the PZR vaporizer well (in place of the cover), a disk component, which sits on top of the modified insert, and a tube device, which fits into the disk component, and from which the vapor is expelled downwardly into the bottle or container. These three components work together to allow a vaporizer device to produce better tasting vapor and distribute the vapor from the vaporizer to a non-proprietary receptacle.

Other configurations of the insert, disk and tube components can be manufactured and still be within the scope of the disclosure. For example, a configuration that combines the insert and disk components into one piece, or a configuration that combines all three components to achieve the same result. Other configurations can be understood, based on this disclosure, to be included within the scope of the present disclosure.

In addition, a container or receptable, such as a glass bottle with a hole in the bottom is required in the present disclosure. The receptacle will be placed over and be securely accepted by the tube device, either tapered or straight with a friction band, before turning on the vaporizer device.

The present disclosure, which describes the Vapor Bottle Nozzle Adapter device in detail, allows a desktop vaporizer device to fill a receptacle, such as a standard glass bottle containing a hole in the bottom, with vapor in such a way that the use of silicone parts in the airpath is minimized or eliminated, and which allows a home user to easily modify existing glass bottles to permit their use as receptacles for such vapor, by drilling a hole in the base of the bottle.

It is an objective of the present invention to develop a vaporizer device adapter, or a Vapor Bottle Nozzle Adapter consisting of non-silicone components, either one, two or three depending on the design. For an embodiment, the three-piece design comprises three main components including an insert component or piece placed at the bottom, a disk component or piece, placed in the middle, and a tube device or piece, placed on top of the disk component. These components work together to channel vapor from the PZR base unit into the receptacle bottle.

It is an objective of the present invention to develop a vaporizer device adapter, or a Vapor Bottle Nozzle Adapter consisting of non-silicone components, either one, two or three depending on the design. For the two-piece design, the insert component is not used, and the original PZR vaporizer device insert component can be used instead. Accordingly, the original cover fits into the well of the vaporizer device, the disk component fits over and around the cover. The tube device then fits into the disk component and protrudes into the receptacle while accepting and securely grasping the lower edge of the hole in the receptacle.

It is an objective of the present invention to develop a vaporizer device adapter, or a Vapor Bottle Nozzle Adapter consisting of non-silicone components, either one, two or three depending on the design, which are made of various materials which do not retain odors, such as glass or metal, or materials that retain pleasant odors, such as wood.

It is an objective of the present invention to develop a vaporizer device adapter, or a Vapor Bottle Nozzle Adapter consisting of non-silicone components, either one, two or three depending on the design, and accepts and securely attaches to many different receptacle or container types, by requiring only a simple hole placed in the bottom of almost any bottle or container, including those receptacles with no heel, a small heel or a tall heel. The hole can be simply drilled into the bottom of any glass container using a diamond hole saw bit. In the preferred embodiment, the hole size can be about 18 mm.

It is an objective of the present invention to develop a vaporizer device adapter, or a Vapor Bottle Nozzle Adapter consisting of non-silicone components, either one, two or three depending on the design, and accepts and securely attaches to many different receptacle or container types, as there are many different types of existing beverage or food bottles and receptacles commonly available, which have interesting characteristics of size, shape and color. Many users of vaporizer devices would like to make their own vapor receptacle bottles from interesting bottles they have or might locate.

It is yet another objective of the present invention to develop a vaporizer device adapter, or a Vapor Bottle Nozzle Adapter consisting of non-silicone components, either one, two or three depending on the design, and accepts and securely attaches to many different receptacle or container types, since existing PZR vaporizer devices come with a glass receptacle with a relatively large 1″-1.25″ opening at the top for the user to inhale the vapor. Some people find uncomfortable to use the standard receptacle. By allowing users to create their own vapor bottles and receptacles, they can make receptacles with more comfortable mouthpieces from existing, easily available beverage bottles.

It is an objective of the present invention to develop a vaporizer device adapter, or a Vapor Bottle Nozzle Adapter consisting of non-silicone components, either one, two or three depending on the design, and accepts and securely attaches to many different receptacle or container types, since the vapor is more dense than plain air, it naturally sinks to the bottom of a receptacle. This property makes it appear to flow like a liquid inside the bottle, but more slowly. When bottles of various shapes are tilted or inverted, the vapor will flow in various ways. When a bottle with a wide midsection or belly is tilted sideways, the vapor will flow into the belly first, and collect there. If a conical bottle is used and pointed downwards, the vapor will flow into the neck of the bottle, concentrating there. At that point, the user can inhale the vapor with very little excess air. There are many other interesting examples of the vapor action when using bottles of different shapes. Of course, there are many other reasons a user might want to make their own vapor bottles, such as for special occasions with custom sizes and printing, or for use with certain substances and for humorous purposes, etc.

If the dimensions of any of the PZR-style vaporizer bases change, or if new PZR-style vaporizers are sold, the dimensions of the present disclosure may likewise need to change to fit any new style or dimensioned vaporizer devices. This is due to the present disclosure, and to the extent that it requires the use of one of the PZR-style vaporizer base units, whereby the disclosure replaces the nozzle and glass receptacle components of these PZR-style devices, utilizing only the base unit. To the extent that the present disclosure utilizes its own base unit, changes in the dimensions may not be required.

Other objects, features and advantages of the disclosure and invention will be apparent from the following detailed disclosure, taken in conjunction with the accompanying sheets of drawings, wherein like reference numerals refer to like parts.

### DRAWINGS

The preferred and alternative embodiments of the disclosure will be described in conjunction with the appended drawings provided to illustrate and not to the limit the disclosure, where like designations denote like elements, and in which:

FIG. 1 shows a perspective view of a prior art vaporizer device in which vapor flows up from base through silicone nozzle, to be collected in a glass receptacle.

FIG. 2 shows a perspective view of an improved vapor bottle nozzle adapter, in accordance with an embodiment of the present disclosure.

FIG. 3 shows a perspective view of an improved vapor bottle nozzle adapter, in accordance with an embodiment of the present disclosure.

FIG. 4A shows a perspective view of an improved assembled vapor bottle nozzle adapter, in accordance with an embodiment of the present disclosure.

FIG. 4B shows a perspective view of an improved vapor bottle nozzle adapter, including the two individual pieces the disk component, and the tube device, in accordance with one embodiment of the present disclosure.

FIG. 4C shows a perspective view of an improved assembled vapor bottle nozzle adapter, in accordance with an embodiment of the present disclosure.

FIG. 4D shows a perspective view of an improved vapor bottle nozzle adapter, including the three individual pieces the insert component, the disk component, and the tube device, in accordance with one embodiment of the present disclosure.

FIG. 5 shows an exploded and cutaway view of an improved vapor bottle nozzle adapter and methods, in accordance with an embodiment of the present disclosure.

FIG. 6A shows a perspective view of an improved vapor bottle nozzle adapter, including a tapered tube device, in accordance with an embodiment of the present disclosure.

FIG. 6B shows a perspective view of an improved vapor bottle nozzle adapter, including a tapered tube device, in accordance with an embodiment of the present disclosure.

FIG. 7A shows an exploded view of an improved vapor bottle nozzle adapter, including a tapered tube device, in accordance with an embodiment of the present disclosure.

FIG. 7B shows an exploded view of an improved vapor bottle nozzle adapter, including a tapered tube device, in accordance with an embodiment of the present disclosure.

FIG. 8A shows a perspective view of an improved vapor bottle nozzle adapter, with insert component only located in vaporizer device, in accordance with an embodiment of the present disclosure.

FIG. 8B shows a perspective view of an improved vapor bottle nozzle adapter, in accordance with an embodiment of the present disclosure.

FIG. 9A shows a perspective view of an improved vapor bottle nozzle adapter, including the two individual pieces the disk component, and the tube device, in accordance with one embodiment of the present disclosure.

FIG. 9B shows a perspective view of an improved vapor bottle nozzle adapter, including the three individual pieces the insert component, the disk component, and the tube device, in accordance with one embodiment of the present disclosure.

FIG. 9C shows a perspective view of an improved vapor bottle nozzle adapter, including the three individual pieces the insert component, the disk component, and the tube device, in accordance with one embodiment of the present disclosure.

FIG. 9D shows a perspective view of an improved vapor bottle nozzle adapter, including the opposite sides of the insert component and the disk component shown in FIG. 9C, in accordance with one embodiment of the present disclosure.

FIG. 9E shows a perspective view of an improved vapor bottle nozzle adapter, including an alternative tube device to those shown in FIGS. 9A, 9B and 9C comprising a straight tube and friction band, in accordance with one embodiment of the present disclosure.

### DETAILED DESCRIPTION

Referring to the drawings, wherein like reference numerals refer to the same or similar features in the various views, the present disclosure pertains to an improved vapor bottle nozzle adapter and methods for improved distribution of vapor from a vaporizer device, including in order to solve the well-known shortcomings in the vaporizer device industry, the improved adaptor provides an interface between the vaporizer device and the container that eliminates any silicone odor and taste and allows the use of existing receptacles and bottles for receiving and storing the vapor to be inhaled.

As such, the present disclosure is for those who want a clean, non-silicone airpath and/or the ability to make or buy a wide variety of receptacles, such as various types of glass bottles or jars.

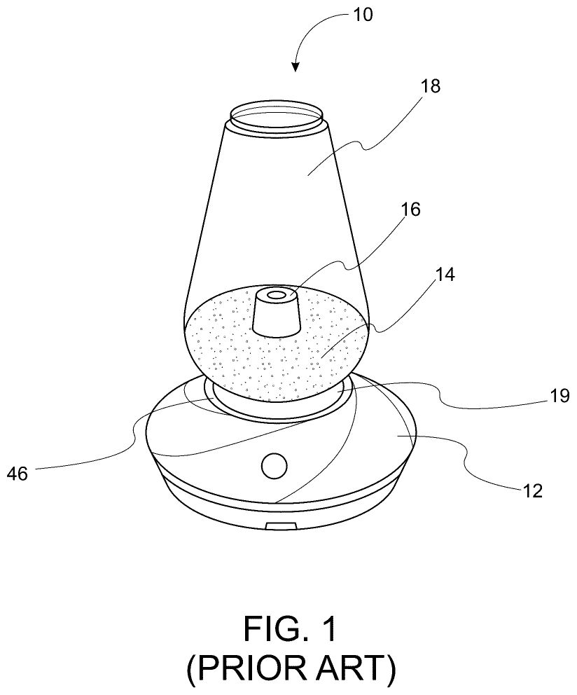

Vaporizer devices exist that are used to push air through the device, expelling vapor into a container, thereby relieving the user of sucking air directly through the vaporizer device. FIG. 1 shows a prior art vaporizer device 10. In general, vaporizer devices 10 consist of a base unit 12 that emits vapor 14 vertically from a base unit through a silicone nozzle 16, into a special matching glass container 18. The vaporizer device 10 also contains a cover 19, which fits inside and protrudes up from the base unit 12. Existing PZR vaporizer devices all operate in a similar fashion, but use differently designed and sized base units 12 and are packaged with various types of glass containers 18, along with other accessories.

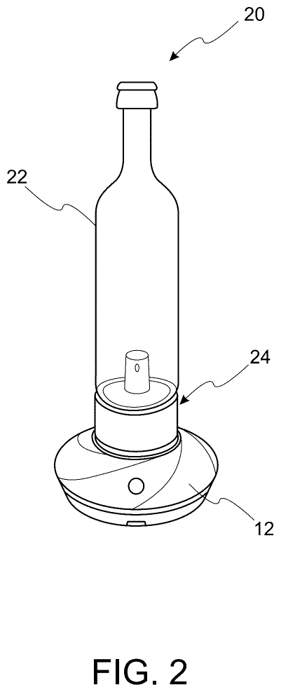

FIGS. 2, 3 and 4A through 4D show perspective views of the preferred and alternative embodiments of the present disclosure. The present disclosure comprises an improved vapor bottle nozzle adapter system 20 for use in the process of producing vapor, from substances such as e-cig vapor or liquid, CBD, and cannabis products, such as flower, oil, concentrates, etc., ultimately for human inhalation.

The disclosure comprises various novel components, which make up the improved vapor bottle nozzle adapter system 20. The components of the improved adapter system 20 comprise the base unit 12, usually taken from an existing prior art vaporizer device 10, although a new base unit can be developed and utilized with the present disclosure.

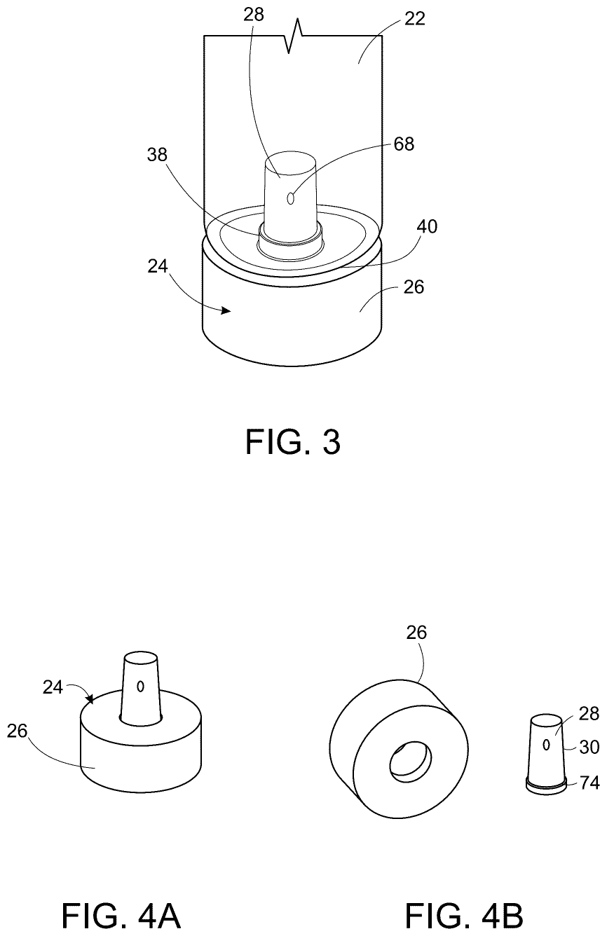

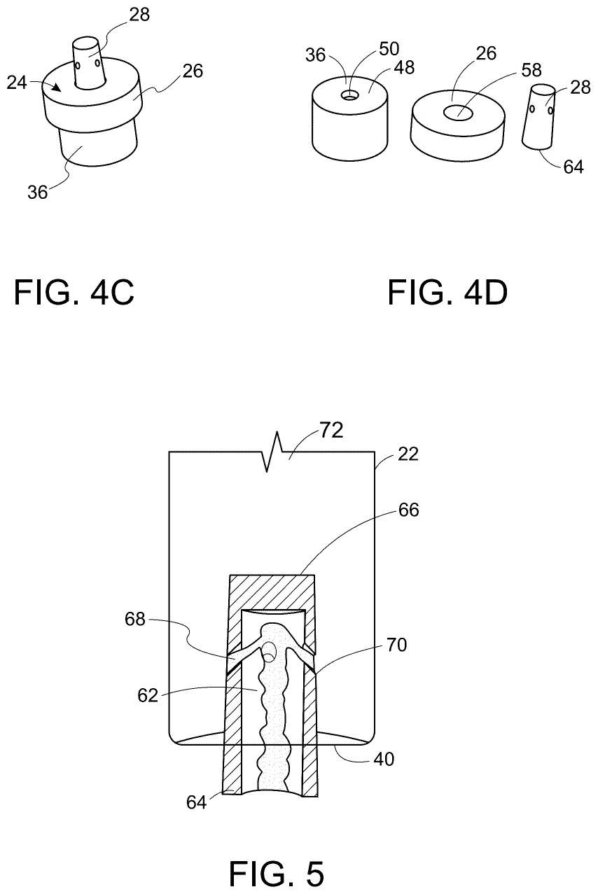

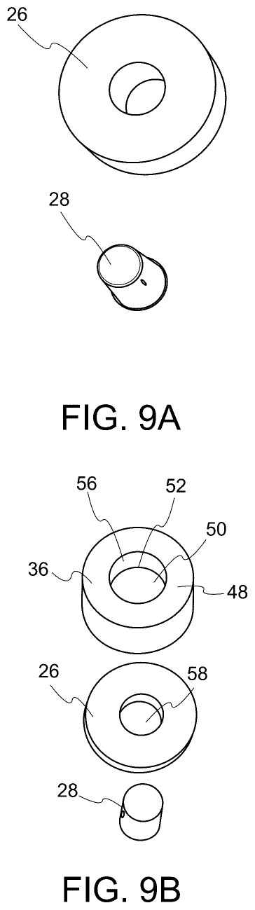

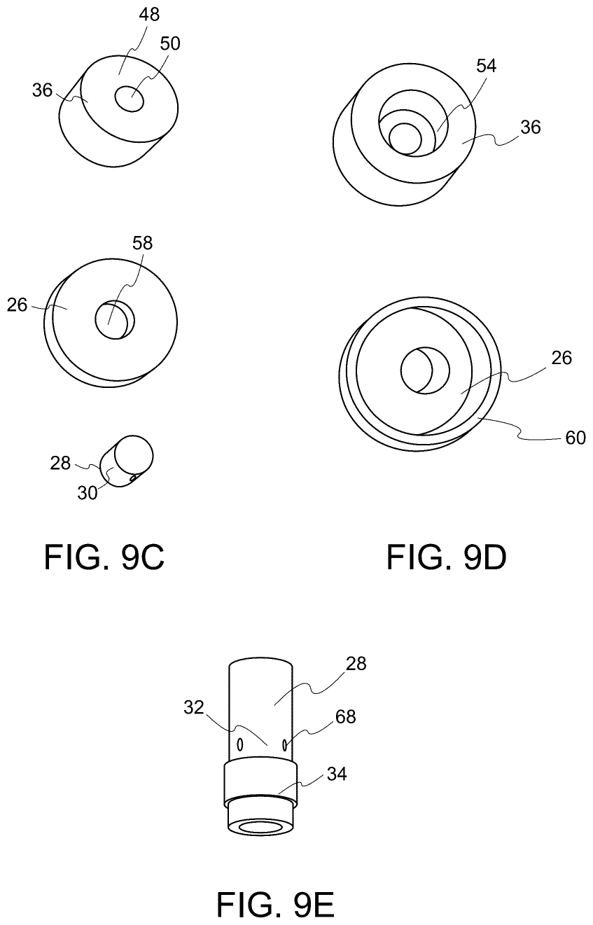

FIG. 3 shows a close-up perspective view of the adapter system 20, which also comprises a modified glass bottle as a receptacle 22, and a novel nozzle assembly 24. The novel nozzle assembly comprises different components depending on the configuration of the nozzle assembly 24. In an embodiment, the nozzle assembly 24 comprises two separate components, a disk component 26 and a tube device 28 (FIGS. 3, 4A and 4B). As detailed herein, the tube device can be tapered 30 or straight 32 utilizing a friction band 34 (FIG. 9E).

In an alternative embodiment, the nozzle assembly 24 comprises three separate components, a disk component 26, a tube device 28 and a modified insert component 36 (FIGS. 4C and 4D). As with the preferred embodiment, the tube device 28 of the alternative embodiment can be tapered 30 or straight 32 utilizing a friction band 34 (FIG. 9E).

The modified receptacle 22 comprises a receptacle hole 38 in the bottom 40 of the modified receptacle 22. As detailed herein, the receptacle hole can be one of multiple configurations and will be based on the size of the tube device 28. This modified receptacle 22 allows for the improved vapor bottle nozzle adapter system 20 to be used for a modified receptacle 22 having a flat or shallow heel 42 (FIG. 7A) or a tall heel 44 (FIG. 7B).

As can be understood, all of these components can and will be sized differently depending on the base unit 12 employed, existing or new, and the size of the receptacle hole 38 in the bottom 40 of the modified receptacle 22.

In particular, for the three-piece nozzle adapter system 20, the modified insert component or piece 36 may be shaped as a cylinder (as shown) or another shape to fit securely inside the well 46 of the base unit 12 of a PZR-style vaporizer device (FIG. 1). The insert component 36 is shaped to allow room for an atomizer or cartridge (not shown), such as a 510 cartridge, which is located in the center of the well 46. The top 48 of the insert component 36 provides a platform for the disk component or piece 26 to rest on. In the middle of the insert component 36 is an insert hole 50 to allow the vapor 14 to travel up out of the insert component 36 and into the tube device 28.

The insert hole 50 in or near the center of the insert component 36 may be of one width or varying widths, as long the insert hole 50 allows room for the atomizer beneath it or a 510 cartridge to stick up through (not shown). Optimally the insert hole 50 is wider at the bottom 52 (FIG. 9 B) to accommodate the atomizer, and narrower at the top 48 to allow less air space in the well 46, and therefore less waste of vapor 14 which does not make it into the modified receptacle 22.

FIGS. 9B and 9D show that the shape of the insert hole 50 can be countersunk 54 or straight 56. The insert hole 50 when countersunk 54 with a smaller diameter hole opening for the tube device 28 tends to work better as it provides a more direct path for the vapor 14 to enter the modified receptacle 22. For the two-piece adapter system 20 design, the insert component is not used, or can be integrated into the disk component.

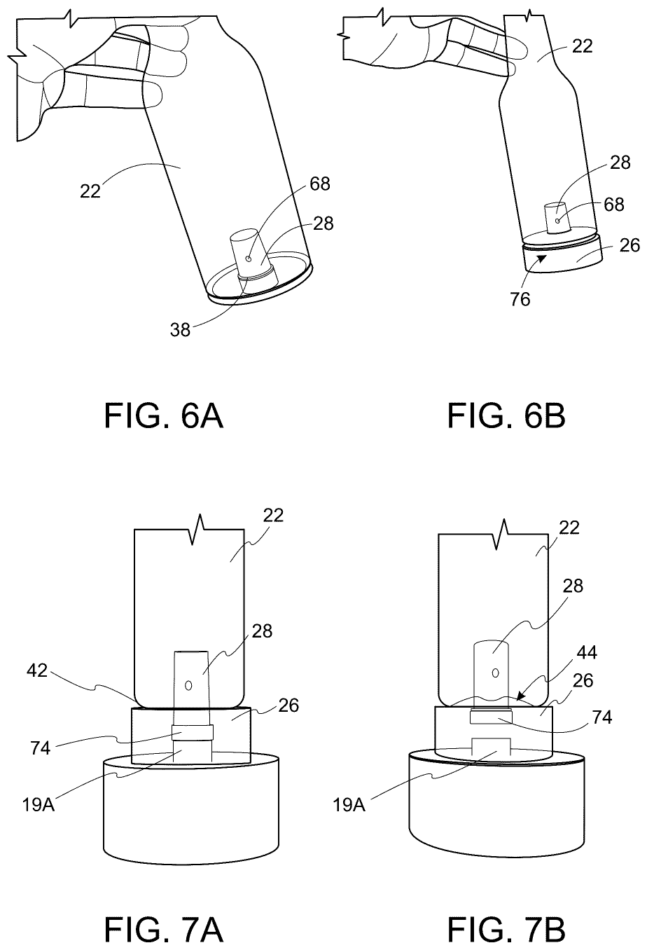

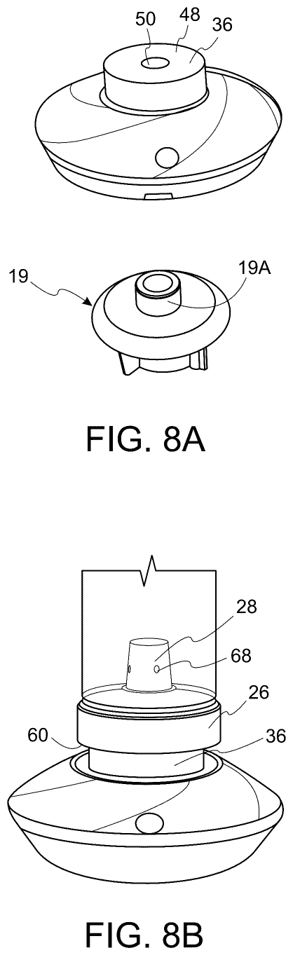

FIGS. 6A, 6B, 7A, 7B, 8A, 8B, and 9A through 9E show components and assemblies of the vapor bottle nozzle adapter system 20. As shown, the disk component 26 sits flush on the insert component 36, when used, or on the original PZR-styled cover 19. The disk component 26 is centered upon the insert component 36 so the hole 58 in the disk component 26 aligns with the insert hole 50. The disk component hole 58 is sized to allow the tube device 28 to fit snuggly therein and ride up and down in that disk component hole 58 like a piston in a cylinder.

The disk component 26 may be centered on the insert component 36 in a variety of ways, such as having the same shape and diameter as the insert component 36, so the two components 26, 36 can be aligned by simply lining them up vertically. Additionally, the disk component 26 may contain a lip 60 which overlaps the insert component 36, thereby aligning them. Also, the disk component 26 may contain grooves, protrusions, or other markings (not shown) to allow such alignment.

For the two-piece nozzle adapter system 20 design, the disk component hole 58 must be slightly wider, to be able to fit over the protruding neck of the original PZR-style cover 19.

FIG. 5 shows an exploded and cutaway view of the tube device 28, which comprises a vertical hole 62 that is open on the bottom of the tube 64 and extends up into the tube for a length, but not through the entire tube, leaving a closed end at the top 66 of the tube device 28. Instead, the tube device comprises tube device expeller holes 68, either straight (not shown) or slanted downward. The tube device expeller holes 68 radiate from the tube device vertical hole 62 to the exterior of the tube device 70, to allow the vapor 14 to be distributed into the modified receptacle 22. The tube device expeller holes 68 are shown at about 45 degrees downward, but any downward facing expeller hole 68 will force the vapor 14 to be distributed towards the bottom 40 of the receptacle 22.

In an embodiment, the expeller holes 68 are angled down towards the bottom 40 of the receptacle 22. This downward angle aims the vapor 14 towards the bottom 40 of the receptacle bottle 22 where it collects and fills the receptacle 22 smoothly from the bottom 40 to the top 72, which is helpful for accurate dosing. If the expeller holes 68 are not slanted, the vaporizer device with the nozzle adapter system 20 will function to emit vapor 14 into the bottle 22, but the vapor 14 will not so readily collect in and fill up from the bottom 40 of the bottle or receptacle 22.

Additionally, the tube device exterior 70 may be either tapered 30 (FIG. 9C) or straight 32 (FIG. 9E). If tapered 30, the tube device 28 is configured as a round piece which is tapered 30 to allow it to protrude into the bottle 22 and grasp the bottom of the bottle 40 at the receptacle hole 38 through friction, so it stays attached to the bottle when the bottle is lifted (see FIG. 6A). The preferred embodiment tube device 28 is tapered from ⅝″ (15.9 mm) at the top 66 (smaller than 18 mm) to ¾″ (19.05 mm) at the bottom 64 (larger than 18 mm), so the tube device 28 will fit into the bottle hole 38, but not all the way through. This configuration allows the bottle 22 to cling to the tapered 30 tube device 28 at the receptacle hole 38.

The tube device base or bottom 64 fits into the hole 58 in the disk component 26 with a snug fit that is tight enough to stay in place, but loose enough for the user to insert and remove the receptacle 22 by hand from the disk component 26. The tube device 28 has a shoulder 74 (FIG. 4B) below the taper 30 to allow the tube device 28 to be pushed in and out of the disk component 26 with a snug fit at any point, much like a piston can slide snugly within a cylinder. This “piston-in-cylinder” sliding mechanism allows for the adapter system 20 to work with bottles 22 with various heel heights 42, 44. This “piston-in-cylinder” sliding mechanism also allows for functioning even if there are small variations in the relative heights of the disk component and tube device.

Alternatively, a non-tapering or straight 32 cylindrical tube device 28 may be used in the nozzle adapter system 20. When sizing the outside diameter of the tube device 28, if it is too big, the tube device 28 will not fit into the 18 mm hole 38 and therefore will not function to fill the bottle 22 with vapor 14.

If the tube device 28 is the correct size of the hole 38, the tube device 28 will fit snuggly, tight enough to grasp the bottle 22 through friction but not too tight to not be easily removed from the bottle 22 by hand. This level of precision is difficult to attain with wood and may only work intermittently. To grasp the inside of the bottle 22 through friction, it requires a semi-soft material such as wood, and very precise sizing which may difficult given the tolerances of wood manufacturing. In addition, variations in air humidity can expand and contract the wood, leading to an improper tube device 28 fit.

If the tube device 28 is smaller than the receptacle hole 38, the tube device 28 will not grasp the inside wall of the 18 mm hole 38 in the bottle 22. To address this issue, a friction band 34 (FIG. 9E) may be applied to encircle the base of the tube device 28, so it fits more snuggly into the 18 mm hole 38. This friction band 34 is made of a flexible material, such as cork, leather, or rubber, among others. However, some such materials may impart an (unpleasant) taste or unhealthful impurities to the vapor.

The friction band 34 requires additional manufacturing complicating production and creates friction on a potentially thin surface which can lead to wear and tear and the need for replacement. To prevent this issue, a pliable material is used such as cork or rubber to make the entire tube device 28 and provide the needed friction effect. However, pliable materials such as these may cause the same taste and health concerns as when they are used for the friction band alone.

Both the tapered 30 or straight 32 designs allow the tube device to remain with the bottle 22 when it is raised (FIG. 6A), along with the disk component 26 which fits snuggly around the tube device 28 (FIG. 6B). This creates an assembly 76 of the components. As such, once the bottle 22 is filled with vapor 14, the user can pick it up and the tube device 28 and disk component 26 go along with it.

This provides an advantage over picking up the prior art vapor-filled bottle 18. The assembly 76 of the tube device 28 and disk component 26 also provides a flat, stable coaster-like base for the vapor-filled bottle 22 to rest on prior to inhaling, thereby exposing less of the glass to potential breakage when placed on a hard surface.

FIGS. 7A and 7B show the piston-in-cylinder design for the tube device 28, which allows for the bottom 40 of receptacle and glass bottles 22 to vary in both the thickness of the material (e.g. glass) and the height of the heel 42, 44, which is the raised part on the base or bottom 40 of a bottle 22, which protrudes up into the bottle 22. Since different bottles 22 have different heel heights 42, 44, the disclosure allows for using these bottles 22 by using the piston-in-cylinder design. The piston-in-cylinder design allows the tube device 28 to slide within insert component 36 to accommodate different heel heights 42, 44. The tube device 28 contains a straight/parallel 32 shoulder section 74 at the bottom 64, to function as a piston which slides up and down in a secure, vertical orientation. When a bottle 22 with a flat or short heel 42 is used, the tube device 28 extends into the disk component 26 a longer distance. When a bottle 22 with a tall heel 44 is used, the tube device 28 extends into the disk component 26 only a short distance. This piston-in-cylinder design permits the use of bottles and receptacles 22 with different heel heights 42, 44. The tube device 28 can be manufactured with different lengths of parallel/straight 32 section to accommodate a wide range of heel heights 42, 44. The shoulder section 74 can be designed in a number of ways, such as integral with and extending from the tube device 28, or as a straight edge at the end of the tapered section 30 of the tube device 28, both of which provide a snug fit inside the disk component hole 58 of the disk component 26.

FIGS. 9A through 9E show the different embodiments of the disk component 26, tube device 28 and insert component 36. The preferred embodiment utilizes the tapered 30 tube device 28 for wedging purposes. As detailed herein, the tube device 28 is tapered 30 to grasp the receptacle hole 38 at the bottom 40 of the bottle 22. The tube device 28 taper 30 starts at ⅝″ (15.9 mm) and increases to ¾″ (19.05 mm). The hole 38 in the bottle 22 is approximately 18 mm, which is in between the ⅝″ (15.9 mm) top and the ¾″ (19.05 mm) bottom of the tube. When placing the bottle 22 over the tube device 28, the 18 mm opening gets stuck about halfway down the tube device 28. This lets the bottle 22 grasp onto the tube device 28 and stay there through strong friction.

Another alternative embodiment utilizes merging of the disk component 26 and the tube device 28 into a single integrated device. If the disk component 26 and the tube device 28 are merged into one piece, the combination or assembly 76 will function to fill the bottle 22 with vapor 14. However, without using multiple components, there will be no piston-in-cylinder action or adjustment available for bottles 22 of different heel heights 42, 44.

This lack of action or adjustment limits the receptacles 22 used to those with a specific heel height 42, 44. Bottles 22 with heels that are too tall 44 may not grasp the tube device 28. Those with heels that are too short 42, may push the tube device out of the bottom 64 on the horizontal disk component portion 26, and therefore be unstable. As there are wide variations of heel heights 42, 44 within standard glass bottles 22, this variation might include packaging or selling of a specific bottle 22 for users, to ensure correct fit. In addition, this one-piece assembly or variation 76 will require the use the cover 19 of the original base unit 12, which does not mitigate the silicon odors from within the base chamber.

As stated herein, preparing these bottles 22 as a vapor receptacle is accomplished by drilling a round 18 mm″ hole in the base of the bottle 22. As one having ordinary skill in the art would understand, this is done with a diamond-tipped hole saw bit mounted in a standard electric drill (not shown). This allows for users to make or use a wide variety of glass receptacles 22, according to their needs or aesthetic preferences.

In the current design, all components can be made from a variety of materials, such as wood, glass, metal, etc. The preferred embodiment utilizes wood because it is easily fashioned, either through CNC machining, or using a wood lathe and drill press, or other ways as understood by one having ordinary skill in the art. Further, using wood or another pliable material that will not leave an unpleasant odor, allows for the edge of the 18 mm hole in the bottom 40 of the glass bottle 22 to be easily pushed onto and grasp the tapered 30 tube device 28 through friction. At the same time, the tube device 28 fits snuggly inside the disk component 26. This allows the bottle 22, tube device 28 and disk component 26 to be picked up together as one unit, providing a minimal seal on the bottom 40 of the bottle 22 to prevent vapor 14 from leaking out, and also provides a coaster “stand” for the bottle to stand upright upon.

The present disclosure permits a wide variety of follow-on products and uses, including components made from various materials (glass, metals, various types of wood), different aesthetic receptacle 22 styles containing ornamentation or engraving for gifts or special occasions, various bottles 22 for various uses, for example, cylindrical bottle with marks on the side—for accurate dosing, small bottle for small draws of vapor, large bottles for sharing of vapor bottle by more than one user, conical bottles for tilting downwards for minimal extra air consumption, bottle with straw in it for sucking vapor from bottom of the bottle, bottle with built-in cap for temporary sealing and passing to another individual, among other devices.

In an embodiment, three component pieces disk component 26, tube device 28, and insert component 36, were crafted of wood based on the current dimensions of the current vaporizer devices, which have a round 2″ wide baseplate. The insert component 36 may be shaped in various ways, depending on the interior shape of the specific vaporizer device and how it is to be used. However, as the current vaporizer devices all have the same round 2″ wide rim on the bottom, a convenient shape for the insert component 36 is a 2″ wide round cylinder of wood which is approximately 1.5″ tall. This shape works in all of the current PZR-style vaporizers. The insert component 36 is shaped as a cylinder which is 2″ wide and 1¼″ tall, with a round hole through the vertical middle of the piece.

This insert piece 36 may be formed in a number of variations. In the simplest implementation, the inside of the insert component 36 with a round exterior of the 2″ cylinder comprises a round hole 50 of approximately 1″ extending through the insert piece 36, forming a short wood pipe with thick walls. This model sits on the round 2″ base plate in the base of the existing base unit 12.

Alternatively, the round hole 50 in the piece is a stepped countersunk hole 54. The first section of the round stepped hole 54 is approximately 1″ tall×1″ wide to accept the bowl in the bottom of the existing PZR-style vaporizer device 12. The second section of the stepped hole is approximately ½″ wide and ¼″ deep, to allow 510 cartridges to still function. Together the two sections form an air passage for the vapor 14 when it is emitted from the PZR-style atomizer bowl or 510 cartridge and allows it to pass through to the tube device 28. This clear air path allows vapor 14 to pass from the PZR bowl unobstructed through the lower piece and shielded from contact with the existing silicone base. This design also takes up more air space than the straight shaft, leaving less space for vapor, which wastes less vapor than the straight shaft above. This model sits on the round 2″ base plate in the base of the existing PZR-style unit 12.

In another alternative embodiment, the hole 50 may be tapered from 1″ wide at the bottom to 0.5″ at the top, to form a conical wooden chamber. This model sits on the round 2″ base plate in the base of the existing PZR-style unit 12.

In the two-piece assembly design 76 containing the tube device 28 and the disk component 26, the insert component 36 is not used.

The disk component 26 can be designed and manufactured in at least three ways. Using straight sides and a round, flat cylinder which is the same diameter as the insert component (2″) and approximately ½″ thick, with a round ¾″ (19.05 mm) hole going all the way through the center. This piece 26 can be easily aligned with the insert component below it, as they are the same diameter.

The disk component 26 can be designed and manufactured using a lip design, with a round, flat cylinder which is approximately 2.5″ wide and ¾″ tall, with a centered, countersunk hole 54 going all the way through. The first section of the countersunk hole 54 is 2″ wide which extends into the disk approximately ⅛″. The remaining section of the countersunk hole 54 is ½″ wide and extends the remaining way through the disk component 26. The first, wide section of the countersunk hole forms a lip 60 on the lower edge of the disk component 26, into which the insert component fits from below. This lip 60 helps to center the disk component 26 on top of the insert and also provides an added measure of stability to the bottle 22.

In the case where the original PZR-style silicone cover 19 is not removed, the disk sits over and above the original cover of complete PZR-style base unit 12. The original silicone cover 19 has a “neck” at the top. In the collar version of the disk component 26, the hole is wide enough to fit loosely over the neck, so the original PZR-style cover 19 makes a platform on which the disk component 26 can sit. In this way, the disk component 26 rests on the cover piece 19 of the vaporizer device 12, encircling the raised vapor opening (neck) without obstructing the vapor 14. The disadvantage of this approach is that it leaves the vapor 14 to accumulate in the original silicone chamber, allowing it to pick up odors from the silicone.

As described herein, the tube device 28 can be designed and manufactured using a tapered cylinder 30, approximately 1.5″ tall. The outside diameter tapers from ⅝″ (15.9 mm) at the top 66 to approximately ¾″ (19.05 mm) wide near the base 64. The hole 38 in the bottle 22 is 18 mm, which is wider than the small top end 66 of the tube (⅝″) 28, and narrower than the large bottom end 64 of the tube device 28. In this way the bottle 22 can slide over the top 66 of the tube device 28 but not to the bottom end 64 of the tube and wedges up and gets caught on the tube device 28 approximately midway along the tube device 28.

At the very bottom of the tube device 28 is a non-tapered/straight section 74 approximately ⅛″ which stays ¾″ (19.05 mm). This small straight section or shoulder 74 forms a snug fit inside the ¾″ (19.05 mm) hole on the disk component 26. This snug fit in the straight section 74 at the bottom 64 allows the tube device 28 to slide up and down within the disk component 26 like a piston in a cylinder. Adjusting the height up and down allows it to accommodate variations in the height of heel section 42, 44 of the bottle 22. For bottles 22 with tall heels 44, the tube device 28 extends a short way into the insert component 36. For bottles 22 with short heels 42, the tube device 28 extends a longer way down into the insert component 36. Without the small shoulder section 74, the tube device 28 will still function but does not fit as snug and securely in the disk hole 58.

The inside dimensions and design of the tube piece 28 comprises a tapered cylinder 30 with a centered hole 62 of approximately ½″ diameter extending from the bottom to a height of approximately 1¼″ up inside the cylinder. This hole 62 is not open at the top end 66. This makes the tapered cylinder 30 into a hollow tube with ⅛″ walls and sealed top end 66. The internal hole 62 may also be tapered in a conical shape from ½″ wide at the bottom of the tube 64 to a smaller width at the top 66 (sealed) end of the tube device 28. The tube device 28 also has one or more smaller expeller holes 68 which radiate out from the main centered hole 62 and are at a downward angle. These smaller holes 68 extend all the way through the sides and make airways for the vapor 14 to escape into the receptacle 22. These smaller holes 68 are positioned about halfway up the tube device 28 on the outside and proceed at an upwards angle into the top of the main hole 68 in center of the tube device 28, so that when the vapor 14 is expelled, it is expelled downward towards the bottom 40 of the glass bottle or other vapor receptacle 22. If the holes 68 are not slanted downward, the device 20 still works, but the vapor 14 does not fill as smoothly from the bottom 40.

During use of the vapor bottle nozzle adapter system 20 the following steps are performed.

The tube device 28 is pushed so that the narrow end 64 of the tube device 28 sits firmly into the 18 mm hole 38 in the bottom 40 of the receptacle 22, until there is friction, and it stays securely in place.

The disk component 26 is attached to the tube device 28 in the bottle 22, by pushing the tube device 28 into the ¾″ (19.05 mm) hole 58 in the disk component 26, until the bottom 40 of the bottle 22 sits on the top of the disk component 26, with the tube device 28 holding them together.

If using three-piece design disclosed herein with the invention's insert component 36, remove the original silicone cover 19 from the PZR-style vaporizer device 12 and replace with the new insert component 36. The insert component 36 is centered on the round base plate in the bottom of the PZR vaporizer 12 and pushed in place. If using the two-piece design 76, do not remove the original PZR cover 19.

Next, position the assembly 76 made up of the tube device 28 and disk component 26, and the connected bottle 22, centered in its resting place on the cover 19 of the base unit 12. With the three-piece design, place it on the original PZR without the cover 19. For the two-piece design, position it centered over the neck of the original PZR cover 19. The invention is now fully positioned.

Turn on the vaporizer device. Vapor 14 will travel up through the insert component 36 (or cover 19) into the tube device 28 and then downward out through small “downspout” holes 68 into the bottom 40 of the receptacle 22. When the receptacle 22 is filled with vapor 14 to the desired level, turn off the PZR-style vaporizer device 12.

If the filled bottle 22 will be passed to another person, a cap or stopper may be used to seal the filled bottle 22 first before it is raised off the insert 19, 36. Then it can be removed from the vaporizer device 12 and easily passed to another person with minimal loss of vapor 14. From the bottle 22 with vapor 14 the user then inhales vapor 14 from the open top 72 of bottle 22. Regardless of whether or not a cap or stopper is used, after lifting the bottle 22 from the base unit 12, the bottle 22 may be tilted downward or upside down to allow the vapor 14, which is denser than air, to flow into the neck of the bottle 22, so the vapor 14 may be inhaled first without inhaling excess air along with the vapor 14, resulting in a smaller draw volume and more comfortable inhalation experience.

It will be understood that the embodiments of the present disclosure, which have been described, are illustrative of some of the applications of the principles of the present disclosure. Although numerous embodiments of this disclosure have been described above with a certain degree of particularity, those skilled in the art could alter the disclosed embodiments without departing from the spirit or scope of this disclosure.

All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosed system and methods.

Additionally, joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosed apparatus, system and methods as disclosed herein.

What is claimed is:

1. A vapor bottle nozzle adapter system for connecting a forced air style vaporizer device to a standard container, comprising:
a disk component, said disk component comprising a disk hole near the disk center, said disk component sized and shaped to sit on top of a cover of said forced air style vaporizer device; and
a tube device, said tube device comprising a shoulder, said tube device shaped and sized to be pushed in and out of the disk hole of the disk component with a snug fit, said tube device comprising a tube device vertical hole near the center of the tube device, said tube device vertical hole starting at a bottom of said tube device and ending before the top of said tube device, said tube device comprising at least one tube device expeller holes, said at least one tube device expeller holes connected to and radiating out from said tube device vertical hole at a downward angle such that a vapor generated by said forced air style vaporizer devices will be forced through said cover, then through said disk component, then through said tube device vertical hole and through said at least one tube device expeller holes into a bottom portion of a standard container.

2. The vapor bottle nozzle adapter system for connecting a forced air style vaporizer device to a standard container according to claim 1, wherein said disk component is 2 inches in diameter and approximately one-half inch high with a disk component hole of about 0.75 inches going all the way through the center.

3. The vapor bottle nozzle adapter system for connecting a forced air style vaporizer device to a standard container according to claim 1, wherein said tube device fits into said disk component in a piston-in-cylinder motion.

4. The vapor bottle nozzle adapter system for connecting a forced air style vaporizer device to a standard container according to claim 3, wherein said piston-in-cylinder motion allows for a connection to a standard container with a low heel.

5. The vapor bottle nozzle adapter system for connecting a forced air style vaporizer device to a standard container according to claim 3, wherein said piston-in-cylinder motion allows for a connection to a standard container with a tall heel.

6. The vapor bottle nozzle adapter system for connecting a forced air style vaporizer device to a standard container according to claim 1, wherein said tube device comprises a vertical hole and at least one expeller hole, said at least one expeller hole facing downward to force the vapor towards the bottom of the container.

7. The vapor bottle nozzle adapter system for connecting a forced air style vaporizer device to a standard container according to claim 1, wherein said at least one expeller hole is facing downward at a 45-degree angle.

8. The vapor bottle nozzle adapter system for connecting a forced air style vaporizer device to a standard container according to claim 1, wherein said tube device is tapered.

9. The vapor bottle nozzle adapter system for connecting a forced air style vaporizer device to a standard container according to claim 1, wherein said tube device is straight with a friction band.

10. A vapor bottle nozzle adapter system for connecting a forced air style vaporizer device to a standard container, comprising:
an insert component, said insert component comprising an insert hole near the insert center, said insert component sized and shaped to fit inside a well of a forced air style vaporizer device, said insert component further sized and shaped to accept a cartridge or bowl manufactured to be placed inside said well;
a disk component, said disk component comprising a disk hole near the disk center, said disk component sized and shaped to sit on top of said insert component; and
a tube device, said tube device comprising a shoulder, said tube device shaped and sized to be pushed in and out of the disk hole of the disk component with a snug fit, said tube device comprising a tube device vertical hole near the center of the tube device, said tube device vertical hole starting at a bottom of said tube device and ending before the top of said tube device, said tube device comprising at least one tube device expeller holes, said at least one tube device expeller holes connected to and radiating out from said tube device vertical hole at a downward angle such that a vapor generated by said forced air style vaporizer devices will be forced through said cover, then through said disk component, then through said tube device vertical hole and through said at least one tube device expeller holes into a bottom portion of a standard container.

11. The vapor bottle nozzle adapter system for connecting a forced air style vaporizer device to a standard container according to claim 10, wherein said insert component is 2 inches in diameter and 1.5 inches high, and said disk hole is 1 inch in diameter.

12. The vapor bottle nozzle adapter system for connecting a forced air style vaporizer device to a standard container according to claim 10, wherein said insert hole is countersunk, having an insert hole on a top of said insert component that is smaller in diameter than the insert hole on the bottom.

13. The vapor bottle nozzle adapter system for connecting a forced air style vaporizer device to a standard container according to claim 10, wherein said disk component comprises a lip.

14. The vapor bottle nozzle adapter system for connecting a forced air style vaporizer device to a standard container according to claim 10, wherein said tube device fits into said disk component in a piston-in-cylinder motion.

15. The vapor bottle nozzle adapter system for connecting a forced air style vaporizer device to a standard container according to claim 14, wherein said piston-in-cylinder motion allows for a connection to a standard container with a low heel.

16. The vapor bottle nozzle adapter system for connecting a forced air style vaporizer device to a standard container according to claim 14, wherein said piston-in-cylinder motion allows for a connection to a standard container with a tall heel.

17. The vapor bottle nozzle adapter system for connecting a forced air style vaporizer device to a standard container according to claim 10, wherein said tube device comprises a vertical hole and at least one expeller holes, said expeller holes facing downward to force the vapor towards the bottom of the container.

18. The vapor bottle nozzle adapter system for connecting a forced air style vaporizer device to a standard container according to claim 10, wherein said at least one expeller hole is facing downward at a 45-degree angle.

19. The vapor bottle nozzle adapter system for connecting a forced air style vaporizer device to a standard container according to claim 10, wherein said tube device is tapered.

20. The vapor bottle nozzle adapter system for connecting a forced air style vaporizer device to a standard container according to claim 10, wherein said tube device is straight.
